(19) United States
(12) Patent Application Publication
(10) Pub. No.: US 2022/0218283 A1
(43) Pub. Date: Jul. 14, 2022

(54) HOLDER FOR A MEDICAL DEVICE

(71) Applicant: Roche Diabetes Care, Inc., Indianapolis, IN (US)

(72) Inventors: Hans List, Oberzent (DE); Frederic Wehowski, Hockenheim (DE)

(21) Appl. No.: 17/708,648

(22) Filed: Mar. 30, 2022

Related U.S. Application Data
(63) Continuation of application No. PCT/EP2020/077160, filed on Sep. 29, 2020.

(30) Foreign Application Priority Data
Oct. 1, 2019 (EP) 19 200 861.3

(51) Int. Cl. A61B 5/00; A61B 5/145; A61M 5/158

(52) U.S. Cl. CPC A61B 5/6831; A61B 5/14503; A61B 2560/063; A61M 5/158; A61M 2005/1586; A61B 5/14532

(57) ABSTRACT

A holder for a medical device has a transcutaneously insertable portion and at least one housing. The holder has at least one mounting device that has a first side and an opposing second side. The mounting device also has a hole reaching from the first side to the second side. The mounting device is configured for being connected to the housing of the medical device. The holder also has two straps that are respectively attached to the mounting device and are fixedly connectable to each other.

### RELATED APPLICATIONS

This application is a continuation of PCT/EP2020/077160, filed Sep. 29, 2020, which claims priority to EP 19 200 861.3, filed Oct. 1, 2019, the entire disclosures of both of which are hereby incorporated herein by reference.

### BACKGROUND

This disclosure teaches a holder for a medical device, a kit and a method for mounting a medical device on a skin site of an extremity of a user. The devices and the method according to this disclosure may mainly allow adherence of a transcutaneous sensor and a housing of the transcutaneous sensor to a skin site of a user or a patient. Further, the devices and the method according to this disclosure may allow adherence of a medication device such as an insulin pump to the skin site of the user or the patient. Other applications are generally feasible.

Delivering medicine to a user, specifically insulin delivery, plays an important role in the prevention and treatment of diseases, in particular in the treatment of diabetes mellitus. Besides using injection pens or syringes, insulin delivery may specifically be performed by using insulin pumps. In particular, a user is generally required to wear the insulin pump on his or her body at all times. Common pumps for delivering medicine, such as for example insulin, comprise one or more medicine reservoirs. As an example, fluid delivery devices are disclosed in WO 2011/046950 A1.

Further, in the field of medical technology and diagnostics, a large number of devices and methods for determining a presence and/or a concentration of one or more analytes in samples, specifically fluid samples, such as body fluids, and/or for determining at least one parameter of a sample are known. For performing fast and simple measurements, several types of test elements are known, which mainly are based on the use of one or more test chemicals, i.e., on the use of one or more chemical substances, one or more chemical compounds or one or more chemical mixtures, adapted for performing a detection reaction for detecting the analyte or determining the parameter. The test chemical often is also referred to as a test substance, a test reagent, a test chemistry or as a detector substance. In addition to so-called spot measurements, in which a sample of a body fluid is taken from a user in a targeted fashion and examined with respect to the analyte concentration, continuous measurements are increasingly becoming established. Thus, in the recent past, continuous measuring of glucose in the interstitial tissue (also referred to as continuous monitoring, CM) for example has been established as another important method for managing, monitoring and controlling a diabetes state. In the process, the active sensor region is applied directly to the measurement site, which is generally arranged in the interstitial tissue, and, for example, converts glucose into electrical charge by using an enzyme (e.g., glucose oxidase, GOD), which charge is related to the glucose concentration and can be used as a measurement variable. Examples of such transcutaneous measurement systems are described in U.S. Pat. No. 6,360,888 B1 or in U.S. Publication No. 2008/0242962 A1. Hence, current continuous monitoring systems typically are transcutaneous systems or subcutaneous systems, wherein both expressions, in the following, will be used equivalently. This means that the actual sensor or at least a measuring portion of the sensor is arranged under the skin of the user.

Commonly, medical pumps and devices configured for determining a presence and/or a concentration of one or more analytes in samples may be configured for attachment to a skin site of the user or the patient. For this purpose, generally, at least one surface of the medical pump and of the device configured for determining a presence and/or a concentration of one or more analytes in samples may comprise at least one adhesive material configured for attachment of the skin site of the user or the patient. Typically, the medical pump and the device configured for determining a presence and/or a concentration of one or more analytes in samples respectively typically stay on the skin site for several days such as for a time period of two to five days or even one or two weeks. As manufacturing costs of the device configured for determining a presence and/or a concentration of one or more analytes in samples are commonly high, such devices are commonly intended to be in use as long as possible, specifically as health care systems commonly only refund equivalent values of a few test strips per day. Commonly, the attractiveness of such devices increases with their ability to replace as many test strips as possible.

In U.S. Publication No. 2011/319733 A1, devices, systems, methods and kits for releasably mounting a medical device on the body or skin of a user are described. Specifically, a structure adapted for mounting a medical device on the body of a host is described. The structure comprises a first portion and a second portion moveable relative to the first portion. The movement of the second portion relative to the first portion causes a release of at least a portion of a medical device from the first and second portions when operatively mounted thereon.

In U.S. Publication No. 2018/0348048 A1, systems and methods for mounting optical sensors in physiological monitoring devices worn by a user to sense, measure, and/or display physiological information are described. Optical sensors may be mounted in the rear face of the device, emit light proximate a targeted area of a user's body, and detect light reflected from the targeted area. The optical sensor may be mounted in a housing or caseback such that at least a portion of the optical sensor protrudes a distance from at least a portion of the housing. The protrusion distance may be adjustable such that a user may achieve a customized fit of the wearable device. Adjustment of the protrusion distance may also result in a customized level of contact and/or pressure between the optical sensor and the targeted area which may, in turn, result in more reliable and accurate sensing of physiological information.

Despite the advantages of state of the art devices, several technical challenges remain. Specifically, many users or patients are not able to take use of medical pumps and devices configured for determining a presence and/or a concentration of one or more analytes in samples because of skin intolerance against the adhesive of a housing of the medical pump or of the device configured for determining a presence and/or a concentration of one or more analytes. Within just a few days or even hours after mounting the medical pump or the device configured for determining a presence and/or a concentration of one or more analytes via the adhesive material on the skin site, the skin site may be irritated. As a consequence, the user or the patient may remove the medical pump or the device configured for determining a presence and/or a concentration of one or more analytes from the skin site although the life time of the medical pump or the device configured for determining a presence and/or a concentration of one or more analytes has not yet expired.

### SUMMARY

This disclosure teaches a holder for a medical device, a kit and a method for mounting a medical device on a skin site of an extremity of a user which at least partially avoid the shortcomings of known devices and methods of this kind and which at least partially address the above-mentioned challenges. Specifically, devices and methods are disclosed which allow for an adherence of a medical device, specifically of a housing of the medical device, to a skin site of a patient or a user and, at the same time a risk of a skin intolerance of the patient or the user against an adhesive of the housing of the medical device can be avoided.

As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one,” “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once. It shall also be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “mounting device,” “strap,” and “housing,” to name just a few, should be interpreted wherever they appear in this disclosure and claims to mean “at least one” or “one or more” regardless of whether they are introduced with the expressions “at least one” or “one or more.” All other terms used herein should be similarly interpreted unless it is made explicit that a singular interpretation is intended.

Further, as used in the following, the terms “preferably,” “more preferably,” “particularly,” “more particularly,” “specifically,” “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

The terms “patient” and “user” as used herein are broad terms and are to be given their ordinary and customary meaning to a person of ordinary skill in the art and are not to be limited to a special or customized meaning. The terms specifically may refer, without limitation, to a human being or an animal, independent from the fact that the human being or animal, respectively, may be in a healthy condition or may suffer from one or more diseases. As an example, the patient or the user may be a human being or an animal suffering from diabetes. However, additionally or alternatively, the invention may be applied to other types of users or patients or diseases.

In a first aspect of this disclosure, a holder for a medical device is disclosed. The medical device comprises at least one transcutaneously insertable portion and at least one housing. The holder comprises at least one mounting device. The mounting device comprises at least one first side and at least one opposing second side. The mounting device further comprises at least one hole reaching from the first side to the second side. The mounting device is configured for being connected to the housing of the medical device. Further, the holder comprises at least two straps. The straps are respectively attached to the mounting device. The two straps are fixedly connectable to each other.

The term “medical device” as used herein is a broad term and is to be given its ordinary and customary meaning to a person of ordinary skill in the art and is not to be limited to a special or customized meaning. The term specifically may refer, without limitation, to an arbitrary device configured for performing at least one therapeutic purpose. The medical device specifically may comprise one component or an assembly of two or more components capable of interacting with each other, such as in order to perform one or more therapeutic purposes, such as in order to perform a medical procedure. The medical device generally may also be referred to as a medical system.

The medical device may be selected from the group consisting of: a medication device for delivering at least one medication to a user; a medical device for detecting at least one analyte in a body fluid. However, other applications may also be feasible.

The medication device for delivering at least one medication such as a drug and/or a therapeutic agent to a user may be used as part of one or more medical treatments. The medication device for delivering at least one medication to a user may exemplarily be selected from the group consisting of: a medical pump, specifically an insulin pump. However, also other embodiments of the medication device may be feasible.

Further, the medical device may be a medical device for detecting at least one analyte in a body fluid. As further used herein, the term “detecting” refers to a process of determining a presence and/or a quantity and/or a concentration of at least one analyte. Thus, the detection may be or may comprise a qualitative detection, simply determining the presence of the at least one analyte or the absence of the at least one analyte, and/or may be or may comprise a quantitative detection, which determines the quantity and/or the concentration of the at least one analyte. As a result of the detection, at least one signal may be produced which characterizes an outcome of the detection, such as at least one measurement signal.

Specifically, the medical device may have a transcutaneous insertable portion. As used herein, the term “transcutaneous insertable portion” refers to a component, e.g., to a part of an arbitrary device, which is adapted to be fully or at least partly arranged within a body tissue of the patient or the user. For this purpose, the transcutaneous insertable portion generally may be dimensioned such that a transcutaneous insertion is feasible, such as by providing a width in a direction perpendicular to an insertion direction of no more than 5 mm, preferably of no more than 2 mm, more preferably of no more than 1.5 mm. The transcutaneous insertable portion may have a length of less than 50 mm, such as a length of 30 mm or less, e.g., a length of 5 mm to 30 mm. It shall be noted, however, that other dimensions are feasible. The transcutaneous insertable portion may fully or partially provide a biocompatible surface, i.e., a surface which, at least during durations of use, do not have any detrimental effects on the user, the patient or the body tissue.

The term “insertable portion” as used herein is a broad term and is to be given its ordinary and customary meaning to a person of ordinary skill in the art and is not to be limited to a special or customized meaning. The term specifically may refer, without limitation, to an arbitrary portion of an element which may be configured to be at least partially insertable into another object such that the insertable portion may be at least partially located under the object or surrounded by an interior of the object. Specifically, the insertable portion may be configured to be at least partially inserted into the body tissue, specifically under the skin of the patient. Therefore, the insertable portion may specifically have an elongate shape with a small cross-section.

Exemplarily, the transcutaneously insertable portion may be selected from the group consisting of: a sensor, preferably an analyte sensor for detecting at least one analyte in a body fluid, preferably a glucose sensor; an infusion cannula, preferably an insulin infusion cannula. However, other embodiments may also be feasible. As further used herein, the terms “sensor” and “analyte sensor” may refer to an arbitrary element which is adapted to be used in a process of detection. The sensor may exemplarily be an electrochemical sensor having at least two electrodes. The sensor may specifically be a glucose sensor.

As outlined above, the transcutaneously insertable portion may be an infusion cannula. The term “infusion cannula” may generally refer to an arbitrary cannula being configured to introduce an infusion, i.e., a liquid substance, specifically a liquid substance comprising a medicine, into the body tissue. Therefore, the infusion cannula may be attached to a reservoir comprising the liquid substance, specifically via an ex vivo proximal end of the infusion cannula.

The transcutaneously insertable portion may have an elongated or round form. Specifically, the transcutaneously insertable portion may have the form of one of a cylinder, specifically of a rounded cylinder. Further, the transcutaneously insertable portion may have a rectangular cross section or a trapezoidal cross-section. The transcutaneously insertable portion may have an outer diameter from 0.25 mm to 1.5 mm, preferably from 0.5 mm to 1 mm, preferably of about 0.65 mm, more preferably of 0.67 mm.

The medical device may be configured to be mounted on a skin site of an extremity of the user. The extremity may be selected from the group consisting of: an arm, specifically an upper arm; a stomach; a shoulder; a back; hip; a leg. Specifically, the extremity may be the upper arm. However, also other applications may be feasible.

The medical device may further comprise at least one component which may be configured to stay outside of the body tissue. Specifically, the medical device may comprise at least one housing. The housing may correspond to the component which may be configured to stay outside of the body tissue. The term “housing” as used herein is a broad term and is to be given its ordinary and customary meaning to a person of ordinary skill in the art and is not to be limited to a special or customized meaning. The term specifically may refer, without limitation, to an element or component having at least one interior space and at least one wall of fully or partially surrounding the at least one interior space and providing protection to the interior space, such as one or more of a mechanical protection or a protection against environmental influences such as one or more of moisture, oxygen or microbial contaminations. The housing may generally be adapted to fully or partially surround and/or receive one or more elements in order to provide one or more of a mechanical protection, a mechanical stability, an environmental protection against moisture and/or ambient atmosphere, a shielding against electromagnetic influences or the like. The housing may also provide a basis for attachment and/or holding one or more further components or elements.

Specifically, the housing may be configured for holding one or more electronic components. Thus, the housing may also be referred to as an electronics unit. Specifically, in case the medical device is a medical device for detecting the analyte in the body fluid, the electronics unit may be configured for one or more of determining and/or controlling a detection of the analyte or transmitting measurement data to another component. The term “detection” generally refers to a process of determining a presence and/or a quantity and/or a concentration of the at least one analyte. Thus, the detection may be or may comprise a qualitative detection, simply determining the presence of the at least one analyte or the absence of the at least one analyte, and/or may be or may comprise a quantitative detection, which determines the quantity and/or the concentration of the at least one analyte. Specifically, the electronics component may be configured for one or more of performing a measurement with the sensor, performing a voltage measurement, performing a current measurement, recording sensor signals, storing measurement signals or measurement data, transmitting sensor signals to another component. Thus, the electronics unit specifically may comprise at least one of: a voltmeter, an ampere meter, a potentiostat, a voltage source, a current source, a signal receiver, a signal transmitter, an analog-digital converter, an electronic filter, a data storage device, an energy storage device. Specifically in case the medical device is a medication device for delivering the medication to the user, the housing may be an infusion kit having a pump for transferring an infusion into the body tissue of the user or the patient. Other embodiments of the housing may be feasible.

The housing may specifically be embodied as a closed electronics unit. The transcutaneously insertable portion may be at least partially enclosed by the housing.

In an embodiment, the housing may comprise at least one mounting unit. The mounting unit may be used as a support of other components of the medical device such as of the transcutaneously insertable portion and/or electronic components.

However, in particular, the housing may not comprise a mounting unit. In this case, the housing may further be configured for direct attachment to the skin site of the user or the patient. Thus, the housing may comprise at least one attachment component which is capable of forming a connection to the skin site, such as at least one adhesive surface and/or at least one adhesive strip or plaster.

The term “holder” as used herein is a broad term and is to be given its ordinary and customary meaning to a person of ordinary skill in the art and is not to be limited to a special or customized meaning. The term specifically may refer, without limitation, to an element that holds another object in a desired position. Specifically, the holder may be configured to fix, specifically to mount the object to a further object. Thus, as a consequence, the object may be mounted on, specifically attached to, the further object via the holder. While the object is held by the holder, a movement or shifting of the object relative to the holder may be prevented at least to a large extent. Thus, by rotating, shifting or conducting other movements of the holder, the object may stay on the holder. The holder may generally be configured to establish an adhesive bond to the object. Thus, the holder may also be referred to as a mount, a mounting support or a support.

The holder may be configured for being wrapped around an extremity, specifically around an arm. As outlined above, the extremity may specifically be the upper arm. The extremity may correspond to a body portion where the medical device shall be placed. The holder may provide a surface formed and dimensioned for receiving the medical device. The straps may be dimensioned to be wrapped around the extremity. The holder may specifically comprise a length of 200 mm to 2000 mm, preferably of 300 mm to 1500 mm, more preferably of 400 mm to 600 mm. Further, the straps of the holder may comprise a width of 10 mm to 300 mm, preferably of 15 mm to 60 mm, more preferably of 20 mm to 40 mm. Other dimensions may be feasible.

The holder may specifically be embodied as an elongate object which may be configured as a surrounding or enclosing structure, surrounding or enclosing the extremity, specifically the upper arm. The holder may specifically be a three-dimensional structure being configured for encompassing the extremity at least partially. A distance between a surface of the extremity, specifically the skin site of the user or the patient, and a surface of the holder may have a constant value. Specifically, the surface of the holder may be configured to be in direct contact with the surface of the extremity. As used herein, the term “at least partially encompass” may generally refer to a situation in which one object A encloses a circumference of another object B at at least one position or section of the object B.

The holder may specifically be a flexible holder. The term “flexible holder” as used herein is a broad term and is to be given its ordinary and customary meaning to a person of ordinary skill in the art and is not to be limited to a special or customized meaning. The term specifically may refer, without limitation, to an arbitrary holder having portions which may be bendable or deformable by forces which usually occur during holding the portions of the holder with both hands and moving the hands relative to each other, such as forces of 10 N or less. Specifically, the flexible holder may be made of or may contain at least one deformable material, such as at least one plastic or malleable material and/or at least one elastic material. The flexible holder may specifically deform reversibly and may return at least partially to its original shape when an applied stress is removed. The holder may specifically be partially flexible. Thus, the holder may have portions with stiff or rigid properties. Specifically, the straps may be at least partially flexible and the mounting device may be embodied as a stiff component which will further be described below in more detail. However, also other embodiments may be feasible.

As outlined above, the holder comprises the mounting device. The term “mounting device” as used herein is a broad term and is to be given its ordinary and customary meaning to a person of ordinary skill in the art and is not to be limited to a special or customized meaning. The term specifically may refer, without limitation, to a device which is configured to mount, specifically to fix or to attach, an arbitrary object to another element, specifically in a desired position. Thus, as a consequence, the object may be mounted on, specifically fixed or attached to, the other element via the mounting device. While the object is held by the mounting device, a movement or shifting of the object may be prevented at least to a large extent. Thus, by rotating, shifting or conducting other movements of the other element, the object may stay on the mounting device. Specifically, the object may refer to the housing of the medical device as described above or as will further be described below in more detail. Further, the other element may refer to the extremity of the user or the patient. Thus, the mounting device may generally be configured to establish an adhesive bond to the housing of the medical device. Further, the mounting device may generally be configured to establish at least one of a form-fit, a force-fit connection or an adhesive bond to the extremity of the user or the patient. Thus, the mounting device may be configured as a connecting element or binding element between the housing of the medical device and the extremity of the user or the patient. Specifically, in a mounted state of the housing of the medical device to the extremity the mounting device may maintain the form-fit, and/or force-fit connection and/or the adhesive bond to the housing and the extremity, respectively. Thus, in the mounted state, the mounting device itself may be fixedly attached to at least one of the housing, the extremity. Also other embodiments may be feasible. The mounting device may be embodied as a component of the holder. Thus, the mounting device may also be referred to as a mounting portion.

As outlined above, the mounting device comprises the first side and the opposing second side. The term “side” as used herein is a broad term and is to be given its ordinary and customary meaning to a person of ordinary skill in the art and is not to be limited to a special or customized meaning. The term specifically may refer, without limitation, to different surfaces forming an outside of an arbitrary object. The terms “first side” and “second side” may be considered as nomenclature only, without numbering or ranking the named elements, without specifying an order and without excluding a possibility that several kinds of first sides and second sides may be present. Further, additional sides such as one or more third sides may be present.

The first side and the second side may specifically respectively refer to longitudinal sides of the holder. Thus, shorter sides of the holder may respectively extend transverse, specifically perpendicular, to the first side and the second side. The second side may specifically refer to a rear side of the holder and the first side may specifically refer to a front side of the holder. In a mounted state of the holder wherein the holder is fixedly connected to an extremity of the user or the patient, the second side may face a skin site of the user or the patient and the first side may face an outer environment of the user of the patient.

Further, the medical device may be attachable to the holder on the first side. The first side of the mounting device may specifically comprise at least one flat surface configured for attachment of the medical device. In turn, the medical device, specifically the housing of the medical device may have at least one flat medical device surface. The flat surface of the first side of the mounting device and the flat medical device surface of the medical device may be configured to be laid up on top of each other. The flat design respectively of the flat surface of the first side of the mounting device and the flat medical device surface of the medical device may specifically lead to a large contact area between the medical device and the holder, specifically between the mounting device of the holder and the housing of the medical device and lead to a stable connection between the medical device and the holder.

Specifically, the flat surface may be a rough surface, specifically a slightly rough surface. This may lead to an improved adherence of the medical device to the holder, specifically of the housing of the medical device to the mounting device of the holder. Specifically, the rough surface may have a roughness of 0.2 μm to 100 μm, preferably of 0.4 μm to 50 μm, more preferably of 1 μm to 10 μm. Alternatively, the flat surface may be a smooth surface and may specifically be manufactured as a single piece. The flat surface may be made of at least one material selected from the group consisting of: polyurethane, neoprene. Also other materials may be feasible. Specifically, the flat surface may be made of at least one material having a Shore hardness A of 60 to 100, preferably of 70 to 90.

As outlined above, the second side of the mounting device may be configured for facing the skin site of the user or the patient. Only optionally, the second side may comprise at least one adhesive material. The adhesive material may be configured for fixing the holder, specifically the mounting device of the holder, to the skin site during insertion of the transcutaneously insertable portion. The adhesive material may be configured to adhere to the skin site for just a few minutes. Specifically, the adhesive material does not need to adhere to the skin site for several days. After insertion, the medical device may be kept in place by the holder, specifically by the straps surrounding the extremity as will further be described below in more detail. Thus, skin irritation can be reduced.

Further, optionally, the second side may be coated with or may comprise at least one of a soft silicon rubber, a dense foamy rubber, neoprene. Thus, a fixation of the holder, specifically of the mounting device of the holder to the skin site during insertion of the transcutaneously insertable portion may be improved.

The mounting device may specifically be embodied as a flat device having a lateral extension exceeding its thickness by at least a factor of 2, at least a factor of 5, at least a factor of 10, or even at least a factor of 20 or more. The mounting device specifically may have a rounded shape. The shape of the mounting device may correspond to a shape of the medical device, specifically to a shape of the medical device defined by a contact area of the medical device which is configured for resting on the mounting device. However, other shapes may be feasible.

The mounting device may have a thickness of 0.1 mm to 2 mm, preferably of 0.3 mm to 1 mm, preferably of 0.4 mm to 0.7 mm, most preferably of 0.5 mm. The term “thickness” may generally refer to a lateral extension of an arbitrary device in one direction. Specifically, the term “thickness” may refer to a lateral extension of the mounting device transverse, specifically perpendicular, to a direction of extension of the mounting device. The thin design of the mounting device may specifically be advantageous as, typically, the medical device is configured for direct attachment of the skin site of the patient or the user via an adhesive material. The thickness may relate to the lateral extension of the mounting device without taking into account the collar which is in one embodiment comprised in the holder.

Further, the mounting device may be at least partially made of a rigid material. The term “rigid material” generally describes a property of an arbitrary solid material of being unable to bend out of shape. Thus, the rigid material may be resistant against deformation independently from an amount of stress which is applied on the rigid material. Thus, a strain may be zero or at least close to zero and a Young's modulus may be >1000 MPa. Thus, a high stability for the medical device may be provided. The mounting device may be made of a thermoplastic and/or duroplastic material, specifically of a material selected from the group consisting of: polycarbonate (PC), polyurethane (PU), polycarbonate-acrylonitrile-butadiene-styrene blends (PC-ABS blends). Also other materials may be feasible. Further, a hard rubber material having a Shore hardness A of at least 90 may be applicable.

As outlined above, the mounting device comprises at least one hole reaching from the first side to the second side. The term “hole” as used herein is a broad term and is to be given its ordinary and customary meaning to a person of ordinary skill in the art and is not to be limited to a special or customized meaning. The term specifically may refer, without limitation, to an opening, a cavity or a hollow space within an arbitrary object. The hole may specifically have a round ground shape. However, other shapes may be feasible. Specifically, the hole may be a through hole. The term “through hole” may generally refer to a hole of an arbitrary object which fully penetrates the object. The object may have at least two opposing sides and the through hole may extend from one of the two sides to the other one of the two sides thereby penetrating each of the two sides. Thus, the through hole may form a connection between the two sides. The hole may specifically be located in a center of the mounting device. Exemplarily, the mounting device may have a circular shape and a position of the hole may correspond to a center of a circle. However, other positions of the hole may also be feasible. The housing of the medical device as described above may comprise at least one opening, specifically at least one through opening. The opening may be positioned on a side of the housing being configured for attachment to the mounting device of the holder. The opening may be configured for releasing the transcutaneously insertable portion from an interior space of the housing to an outer environment of the housing. In a mounted state of the medical device on the holder, specifically on the mounting device, the opening of the housing of the medical device and the hole of the mounting device may be arranged congruently. The hole of the mounting device may be configured for guiding the transcutaneously insertable portion from the first side of the mounting device to the second side of the mounting device. Thus, the transcutaneously insertable portion may penetrate the skin site of the user or the patient.

As outlined above, the mounting device is configured for being connected to the housing of the medical device. The term “configured for being connected to” as used herein is a broad term and is to be given its ordinary and customary meaning to a person of ordinary skill in the art and is not to be limited to a special or customized meaning. The term specifically may refer, without limitation, to a property of an object to be placeable into and fixable in close physical proximity to another object. In particular, the object that is configured for being connected to another object may be in direct or indirect physical contact with the other object when it is connected to it. Specifically, the housing of the medical device, may have the attachment component which is capable of connecting the housing to the skin site, such as the at least one adhesive surface and/or the at least one adhesive strip or plaster. The adhesive surface of the housing may be configured for attachment to the mounting device of the holder, specifically to the first side of the holder. Thus, the mounting device and the housing may be connected to each other.

Specifically, the holder may comprise at least one collar. The term “collar” as used herein is a broad term and is to be given its ordinary and customary meaning to a person of ordinary skill in the art and is not to be limited to a special or customized meaning. The term specifically may refer, without limitation, to a portion of an arbitrary object which may surround the object in at least two dimensions. The collar may form a protrusion on the object. Thus, the collar may protrude from a surface of the object.

The collar may be arranged on the first side of the mounting device. Specifically, the collar may form a protrusion on the first side. In a cross-sectional view transverse, specifically perpendicular, to the direction of extension of the mounting device the collar may define a cross-sectional profile. The cross-sectional profile may be spherically shaped or may be shaped as an oval. However, other shapes may be feasible. The collar may have a height of 0.5 mm to 6 mm, preferably of 1 mm to 4 mm, more preferably of 1.5 mm to 3 mm. The height may be dependent on dimensions of the medical device. The term “height” may refer to a distance between the surface of the mounting device and a point of the collar being furthest from surface of the mounting device the cross-sectional profile.

The term “strap” as used herein is a broad term and is to be given its ordinary and customary meaning to a person of ordinary skill in the art and is not to be limited to a special or customized meaning. The term specifically may refer, without limitation, to an arbitrary elongate element of a flexible material. Specifically, the strap may be embodied as a strip. The strap may be configured for fastening or holding things together. Further, the strap may be configured to form a looped band either by itself or together with other straps and/or elements. However, other applications may be feasible. Further, specifically, the strap may be embodied as a surrounding or enclosing structure. The straps may specifically be a three-dimensional structure encompassing an object at least partially. A distance between a surface of the object and a surface of the strap may have a constant value. Specifically, the surface of the strap may be in direct contact with the surface of the object.

As outlined above, the straps are respectively attached to the mounting device. The term “being attached to” as used herein is a broad term and is to be given its ordinary and customary meaning to a person of ordinary skill in the art and is not to be limited to a special or customized meaning. The term specifically may refer, without limitation, to a state of at least one object and at least one other object wherein the object and the other object are in contact with each other in such a way that the object does not come off the other object or vice versa without exerting additional forces to the object or the other object in order to remove these objects from each other. Specifically, the straps may respectively be firmly attached to the mounting device. Thus, the straps and the mounting device may respectively be made of different materials. However, other embodiments may be feasible.

At least one of the two straps may be an elastic strap. Thus, exemplarily, at least one of the two straps may be made of at least one elastic material. The term “elastic” may generally refer to a property of an arbitrary object being made of a material of being deformable by up to at least 10%, at least 25%, at least 50% or at least 75%, at least 100%, or at least 150% of a dimension, such as a length of at least one section of the object when stretched parallel to a surface with a force of up to 100 Newton, up to 50 Newton or up to 10 Newton, preferably between 10 to 50 Newton. Thereby, the object may return to an original size and shape when the force is removed. The elastic material may be selected from the group consisting of: a silicone polymer; a silicone copolymer; an elastomer comprising at least one silicone copolymer; a rubber material, specifically neoprene; polyurethane. However, other materials may be feasible. Specifically, the elastic properties of the straps may be achievable by a stretchable material or by a combination of limited resilience of the material and a shape that supports a prolongation of the straps in one direction under load, e.g., via a sinuous cross section.

Further, specifically, at least one of the two straps may be made of at least one foamed material. The term “foamed material” may generally refer to an arbitrary material which is formed by trapping pocket of gas in the material, which may specifically be a solid material. The foamed material may have closed cells and/or open cells. The closed cells may form discrete pockets, each completely surrounded by the solid material. The open cells may be connected to each other. Thus, liquid may flow through a structure formed by the open cells. Preferably, the foamed material may have open cells. The open cells may have an average size of 0.01 mm to 1 mm, preferably of 0.1 mm to 0.5 mm. However, also other dimensions may be feasible. Thus, sweat of the user or the patient or other fluids such as water may be absorbed and an effect on a slip resistance of the straps may be avoided.

The foamed material may comprise at least one material selected from the group consisting of: a silicone polymer; neoprene; polyurethane. However, other materials may be feasible. Also woven or unwoven fibers can serve as bendable nonetheless load carrying material.

As outlined above, the two straps are fixedly connectable to each other. The term “connectable” as used herein is a broad term and is to be given its ordinary and customary meaning to a person of ordinary skill in the art and is not to be limited to a special or customized meaning. The term specifically may refer, without limitation, to a property of an object to be placeable into and fixable in close physical proximity to another object. In particular, the object that is connectable to another object may be in direct or indirect physical contact with the other object when it is connected to it. The term “fixedly connectable” as used herein is a broad term and is to be given its ordinary and customary meaning to a person of ordinary skill in the art and is not to be limited to a special or customized meaning. The term specifically may refer, without limitation, to a state of at least one object and at least one other object wherein the object and the other object are in contact with each other in such a way that the object does not come off the other object or vice versa without exerting additional forces to the object or the other object in order to remove these objects from each other.

Further, the two straps may be releasably connectable to each other. As further used herein, the term “releasable,” in the context of a mechanical connection, refers to the fact that the mechanical connection may be brought from a disconnected state, also referred to as a non-mated state, into a connected state, also referred to as a mated state, and back into the disconnected state at least once, preferably several times. Thus, the mechanical connection may be closed and released at will. Specifically, the mechanical connection may be releasable without using any tools, simply by manual action. As an example, for opening a connection between the two straps forces of no more than 50 N, such as of no more than 20 N, such as of no more than 10 N, may be required. The force may be applied by one hand or even the fingers or fingertips of the user or the patient.

The at least two straps may specifically be fixedly connectable to each other by at least one of a button, a snap, a hook-and-eye-closure, a magnetic locking, a side release buckle, a zipper. However, other closing mechanism may be feasible. Further, a length of at least one of the two straps may be adjustable. Thus, the holder may be adaptable to a circumference of the extremity of the user or the patient.

In a further aspect of this disclosure, a kit is disclosed. The kit comprises the holder as described above or as will further be described below in more detail. The kit further comprises the medical device having at least one transcutaneously insertable portion. For details of the medical device and the transcutaneously insertable portion, reference is made to the description of the medical device above.

Specifically, the medical device may be the medication device having the infusion cannula as described above. Thus, the kit may be an infusion kit. The term “infusion kit” may refer to an assembly of components which are required for a conduction of an arbitrary infusion. Thus, besides of the infusion cannula, the infusion kit may further comprise at least one fluid coupling for coupling the infusion kit to at least one medication device, preferably to at least one medication pump. Thus, the infusion kit may correspond to or may comprise the housing as described above or as will further be described below in more detail.

As described above, the housing of the medical device may comprise the at least one adhesive material. The adhesive material may be configured for attachment of the medical device directly to the skin site of the user or the patient. Further, the adhesive material may be configured for attachment of the medical device to the mounting device of the holder. The housing of the medical device may at least partially be made of a rigid material.

Further, the kit may comprise at least one inserter unit. The term “inserter unit” (also referred to herein as “inserter”) may generally refer to an arbitrary object which is configured for inserting an element into a body tissue of a user or a patient. After the process of insertion, the inserter unit, or at least one component of the inserter unit, may stay within the body tissue of the user or the patient or may be removed from the skin site. Specifically, the inserter unit may have an insertion cannula. The term “insertion cannula” may generally refer to an arbitrary element which may be insertable into the body tissue of the user, particularly in order to deliver or to transfer a further element. The further element may specifically be the transcutaneously insertable portion. Therefore, the insertion cannula may specifically be or may comprise a hollow tube or a hollow needle. Further, the insertion cannula may be one of a slotted cannula, a punched cannula, a cannula having a curved shape. Also other embodiments may be feasible. The insertion cannula may be configured to be inserted vertically or at an angle of 90° to 30° to the body tissue of the user or the patient, wherein an angular point of the angle corresponds to a puncture site of the insertion cannula on the body tissue. However, also other embodiments may be feasible. Specifically, the medical device may be received in the inserter unit. More specifically, the medical device may be enclosed by the inserter unit at least partially. However, also other embodiments may be feasible.

The collar as described above may have a shape which corresponds to a shape of the inserter unit. Specifically, the collar and the inserter unit, specifically a bottom part of the inserter unit, may have a complementary geometric shape which fit into one another. The mounting device, specifically the first side of the mounting device surrounded by the collar, may have a shape which corresponds to a shape of the inserter unit, specifically to a shape of the bottom part of the inserter unit. Exemplarily, the collar may enclose a circular shape and the inserter unit may have a circular shape as well. However, other shapes may be feasible. However, an average diameter of an area enclosed by the collar may be larger than the attachment surface of the medical device such that the medical device may fit into the mounting device. The collar may be dimensioned such that the inserter unit may only have little play within the holder, specifically within the collar. Thus, the collar may be configured for centering the transcutaneously insertable portion of the medical device. The collar may be configured to facilitate a centric alignment of the inserter unit. Thus, an undesired puncturing though the mounting device may be avoided.

In a further aspect of this disclosure, a method for mounting a medical device comprising at least one transcutaneously insertable portion on a skin site of an extremity of a user is disclosed.

The method comprises the method steps as given in the independent claims and as listed as follows. The method steps may be performed in the given order. However, other orders of the method steps are feasible. Further, one or more of the method steps may be performed in parallel and/or on a timely overlapping fashion. Further, one or more of the method steps may be performed repeatedly. Further, additional method steps may be present which are not listed.

The method comprises the following steps:
a) mounting the holder as described above or as will further be described below in more detail on the skin site of the extremity of the user by fixedly connecting the two straps with each other;
b) placing an inserter unit comprising the medical device on the first side of the mounting device such that an end of the transcutaneously insertable portion and the hole are congruent with each other; and
c) inserting the transcutaneously insertable portion into the skin site through the hole of the mounting device.

For further details on the medical device, the transcutaneously insertable portion and the inserter unit, reference is made to the description above.

The medical device may be placed on the first side of the mounting device of the holder such that the adhesive surface of the medical device faces the first side of the mounting device of the holder. Specifically, in step b) the medical device may be placed on the mounting device such that the medical device touches the first side of the mounting device. Alternatively, in step b), the medical device may be arranged in a distance to the mounting device. The medical device and the inserter unit may be placed on the mounting device separately. Exemplarily, the medical device may be placed on the mounting device before the inserter unit is placed on the mounting device. However, alternatively, the medical device and the inserter unit may be commonly placed on the mounting device.

In step b), the inserter unit may be placed on the mounting device such that the inserter unit is enclosed by the collar. During step c), an insertion mechanism of the inserter unit may be conducted. In case the medical device may be arranged in a distance to the mounting device. The mounting device may be moved towards the first side. Further, the inserter unit may be removed from the mounting device after insertion of the transcutaneously insertable portion into the skin site.

The housing may comprise the at least one opening, specifically at least one exit spot, for the transcutaneously insertable portion. The medical device may be placed on the first side of the mounting device such that the exit spot lies in the center of the hole.

The proposed holder for a medical device, the proposed kit and the proposed method for mounting a medical device on a skin site of an extremity of a user provide many advantages over known devices and methods. Thus, specifically, the devices and method according to this disclosure allow for an adherence of a medical device, specifically of a housing of the medical device, to a skin site of a patient or a user while, at the same time, a risk of a skin intolerance of the patient or the user against an adhesive of the housing of the medical device is avoided.

Specifically, the straps may be made of a slip-resistant material. Further, the straps may be made of a material having a good inherent elasticity. Further, a length of the straps may be adjustable. The holder may be mounted on the extremity such that a shifting of the holder may be avoided.

The medical device may comprise the at least one exit spot for the transcutaneously insertable portion and the medical device may be placed on the first side of the mounting device such that the exit spot lies in the center of the hole. For this purpose, the mounting device may comprise the collar. The inserter unit may fit into the collar only with very little play.

After the inserter unit has been triggered and the transcutaneously insertable portion has been placed into the skin site of the user or the patient, the inserter unit may be removed. The housing may remain on the mounting device of the holder, but not directly on the skin site of the user or the patient.

The straps may be made of the foamed material such as made from a silicone polymer, neoprene, polyurethane. Thus, sweat of the user or the patient may be absorbed and an effect on a slip resistance of the straps may be avoided.

The mounting device may be embodied very thin. Specifically, the mounting device may have a thickness of 0.5 mm as the medical devices are configured for direct attachment of the skin site of the patient or the user. In order to provide a sufficient stability, the mounting device may be made of a rigid material such as of a hart plastic material.

Further, the collar may be dimensioned such that a stability of the medical device may be provided. The straps may be tensioned on the extremity in a soft manner. Thus, the skin may intend to bulge outwardly. However, this may be prevented or at least diminished by the stiff housing of the medical device. As a consequence, the skin may be pressed against the soft straps and a safe fit of the sensor may be enabled.

Summarizing and without excluding further possible embodiments, the following embodiments may be envisaged:

Embodiment 1: A holder for a medical device, wherein the medical device comprises at least one transcutaneously insertable portion and at least one housing, wherein the holder comprises:
at least one mounting device, wherein the mounting device comprises at least one first side and at least one opposing second side, wherein the mounting device further comprises at least one hole reaching from the first side to the second side, wherein the mounting device is configured for being connected to the housing of the medical device; and
at least two straps, wherein the straps are respectively attached to the mounting device, wherein the two straps are fixedly connectable to each other.

Embodiment 2: The holder according to embodiment 1, wherein the hole is a through hole.

Embodiment 3: The holder according to any one of embodiments 1 to 2, wherein the first side of the mounting device comprises at least one flat surface configured for attachment of the medical device.

Embodiment 4: The holder according to any one of embodiments 1 to 3, wherein the mounting device has a thickness of 0.1 mm to 2 mm, preferably of 0.3 mm to 1 mm, most preferably of 0.5 mm.

Embodiment 5: The holder according to any one of embodiments 1 to 4, wherein the mounting device is at least partially made of a rigid material.

Embodiment 6: The holder according to any one of embodiments 1 to 5, wherein the mounting device is made of a thermoplastic material.

Embodiment 7: The holder according to any one of embodiments 1 to 6, wherein the second side of the mounting device is configured for facing a skin site.

Embodiment 8: The holder according to any one of embodiments 1 to 7, wherein the second side comprises at least one adhesive material.

Embodiment 9: The holder according to any one of embodiments 1 to 8, wherein the first side is configured for attachment of the medical device.

Embodiment 10: The holder according to any one of embodiments 1 to 9, wherein the mounting device comprises at least one collar.

Embodiment 11: The holder according to embodiment 10, wherein the collar forms a protrusion on the first side.

Embodiment 12: The holder according to any one of embodiments 10 to 11, wherein the collar is a circumferential collar.

Embodiment 13: The holder according to any one of embodiments 10 to 12, wherein the collar is configured for centering the transcutaneously insertable portion of the medical device.

Embodiment 14: The holder according to any one of embodiments 10 to 13, wherein the collar has a shape which corresponds to a shape of the housing of the medical device.

Embodiment 15: The holder according to any one of the preceding claims, wherein the holder is configured for being wrapped around an extremity, specifically around an arm.

Embodiment 16: The holder according to any one of embodiments 1 to 15, wherein a length of at least one of the two straps is adjustable.

Embodiment 17: The holder according to any one of embodiments 1 to 16, wherein at least one of the two straps is an elastic strap.

Embodiment 18: The holder according to any one of embodiments 1 to 17, wherein at least one of the two straps is made of at least one material selected from the group consisting of: a silicone polymer; a silicone copolymer; an elastomer comprising at least one silicone copolymer, a rubber material, specifically neoprene; polyurethane.

Embodiment 19: The holder according to any one of embodiments 1 to 18, wherein at least one of the two straps is made of at least one foamed material, wherein the foamed material comprises at least one material selected from the group consisting of: a silicone polymer; neoprene; polyurethane.

Embodiment 20: The holder according to any one of embodiments 1 to 19, wherein the at least two straps are fixedly connectable to each other by at least one of a button, a snap, a hook-and-eye-closure, a magnetic locking, a side release buckle, a zipper.

Embodiment 21: The holder according to any one of embodiments 1 to 20, wherein the straps and the mounting device are manufactured as one single piece.

Embodiment 22: A kit, wherein the kit comprises the holder according to any one of embodiments 1 to 22, wherein the kit further comprises a medical device having at least one transcutaneously insertable portion.

Embodiment 23: The kit according to embodiment 22, wherein the medical device is selected from the group consisting of: a medication device for delivering at least one medication to a user; a medical device for detecting at least one analyte in a body fluid.

Embodiment 24: The kit according to any one of embodiments 22 to 23, wherein the medical device is a transcutaneous medical device.

Embodiment 25: The kit according to any one of embodiments 22 to 23, wherein the housing of the medical device comprises at least one adhesive material.

Embodiment 26: The kit according to embodiment 25, wherein the adhesive material is configured for attachment of the medical device to the mounting device of the holder.

Embodiment 27: The kit according to any one of embodiments 22 to 26, wherein the housing of the medical device is at least partially made of a rigid material.

Embodiment 28: The holder according to any one of embodiments 22 to 27, wherein the transcutaneously insertable portion is selected from the group consisting of: a sensor, preferably a glucose sensor; an infusion cannula, preferably an insulin infusion cannula.

Embodiment 29: A method for mounting a medical device comprising at least one transcutaneously insertable portion on a skin site of an extremity of a user, the method comprising the following steps:
a) mounting the holder according to any one of embodiments 1 to 21 on the skin site of the extremity of the user by fixedly connecting the two straps with each other;
b) placing an inserter unit comprising the medical device on the first side of the mounting device such that an end of the transcutaneously insertable portion and the hole are congruent with each other; and
c) inserting the transcutaneously insertable portion into the skin site through the hole of the mounting deice.

Embodiment 30: The method according to embodiment 29, wherein the medical device is placed on the first side of the mounting device via the inserter unit, wherein the inserter unit is removed from the mounting device after insertion of the transcutaneously insertable portion into the skin site.

Embodiment 31: The method according to any one of embodiments 29 to 30, wherein the housing comprises at least one exit spot for the transcutaneously insertable portion wherein the medical device is placed on the first side of the mounting device such that the exit spot lies in the center of the hole.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIGS. 1A to 1C show an exemplary embodiment of a holder for a medical device according to this disclosure.

### DESCRIPTION

The embodiments described below are not intended to be exhaustive or to limit the invention to the precise forms disclosed in the following description. Rather, the embodiments are chosen and described so that others skilled in the art may appreciate and understand the principles and practices of this disclosure.

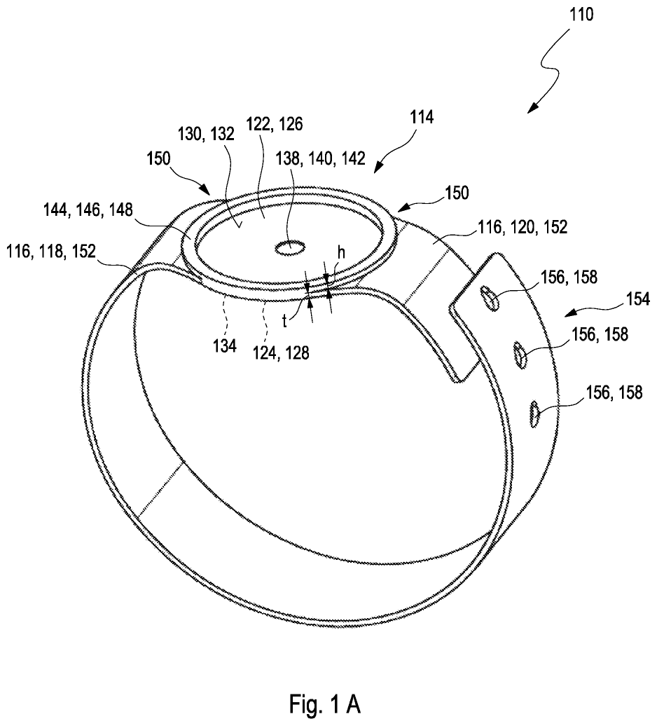

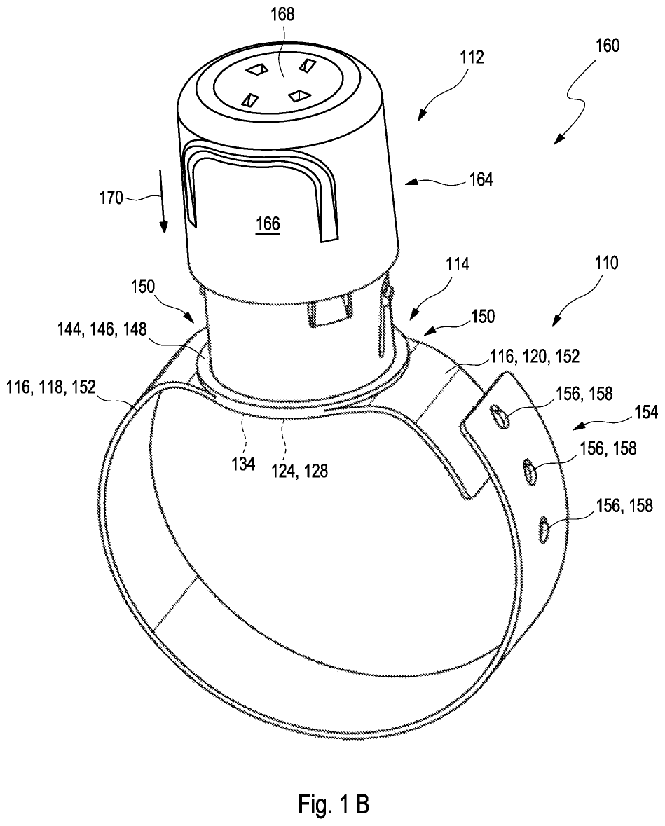

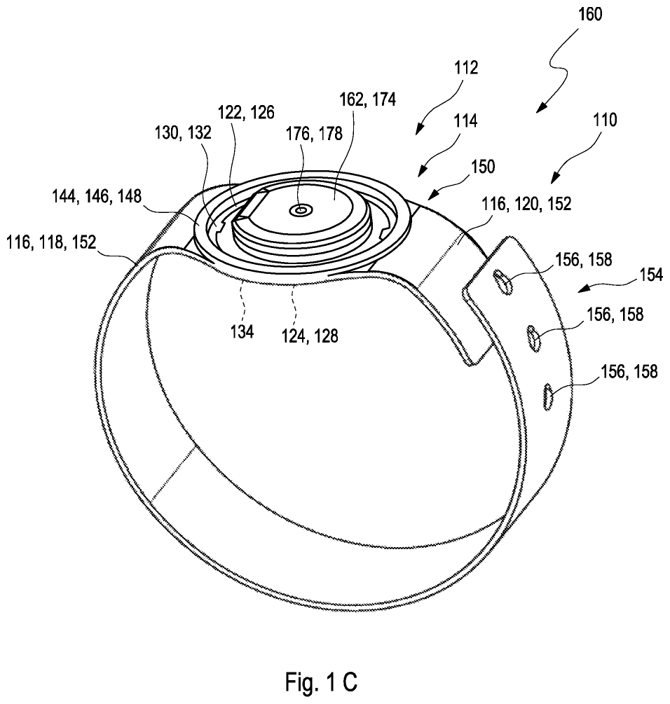

FIG. 1A shows an exemplary embodiment of a holder 110 for a medical device 112 according to this disclosure. The holder 110 together with the medical device 112 is depicted in FIGS. 1B and 1C.

As specifically illustrated in FIG. 1A the holder 110 comprises at least one mounting device 114 and at least two straps 116, specifically at least one first strap 118 and at least one second strap 120.

The mounting device 114 comprises at least one first side 122 and at least one opposing second side 124. In a mounted state of the holder 110, wherein the holder 110 is fixedly connected to an extremity of a user or a patient (not shown), the first side 122 may face an outer environment of the user of the patient and the second side 124 may face a skin site of the user or the patient. Thus, the first side 122 may be a front side 126 of the mounting device 114 and the second side 124 may be a rear side 128 of the mounting device 114. The first side 122 may be configured for attachment of the medical device 112. Thus, the first side 122 may comprise at least one flat surface 130 configured for attachment of the medical device 112. Further, specifically, the flat surface 130 may be a rough surface 132. As a consequence, an adherence of the medical device 112 to the holder 110 may be improved. Optionally, the second side 124 may comprise an adhesive material 134 configured for fixing the mounting device 114 to the skin site during inserting a transcutaneously insertable portion of the medical device 112. The adhesive material 134 may be configured to adhere to the skin site for just a few minutes.

The mounting device 114 may specifically have a rounded shape. The mounting device 114 may have the flat surface 130 as described above. The mounting device 114 may have a thickness t of 0.1 mm to 2 mm, as illustrated in FIG. 1A.

Further, the mounting device 114 comprises at least one hole 138 reaching from the first side 122 to the second side 124. Specifically, the hole 138 may be a through hole 140. Exemplarily, the mounting device 114 may have a circular shape and a position of the hole 138 may correspond to a center 142 of a circle. However, other positions of the hole 138 may also be feasible. The hole 138 of the mounting device 114 may be configured for guiding the transcutaneously insertable portion from the first side 122 to the second side 124. Thus, the transcutaneously insertable portion may penetrate the skin site of the user or the patient. The hole 138 may specifically have a round basic shape. However, also other shapes may be feasible.

Optionally, the holder 110 may comprise at least one collar 144. The collar 144 may form a protrusion 146 on the first side 122. The collar 144 may have a height h of 1 mm to 100 mm. However, also other dimensions may be feasible. Specifically, the collar 144 may be a circumferential collar 148 defining a closed line on the mounting device 114.

The first strap 118 and the second strap 120 may be respectively attached to the mounting device 114. Specifically, the first strap 118 and the second strap 120 may be arranged on opposing sides 150 of the mounting device. The first strap 118 and the second strap 120 may be respectively embodied as a strip 152. The first strap 118 and the second strap 120 may be configured to form a looped band 154 with each other. The first strap 118 and the second strap 120 may be fixedly connectable to each other. Exemplarily, as illustrated in FIG. 1A, the first strap 118 may have at least one hole 156, specifically at least one through hole 158. The second strap 120, in turn, may comprise a protrusion such as a hook (not shown). The protrusion may be configured to be hooked into the hole 156 thereby forming a connection between the first strap 118 and the second strap 120. Specifically, the first strap 118 may comprise a plurality of the holes 156 arranged in one row. Thus, the holder 110 may be adaptable to a circumference of the extremity of the user or the patient.

FIG. 1B shows the holder 110. With regard to details on the holder 110 reference can be made to the description of FIG. 1A above. Further, in FIG. 1B, the medical device 112 is depicted. Specifically, a kit 160 is shown. The kit 160 comprises the holder 110 and the medical device 112. Further, the kit 160 may comprise at least one inserter 164. The inserter 164 may be configured for inserting the transcutaneously insertable portion into the body tissue of the user or the patient. After the process of insertion, the inserter 164 may be removed from the mounting device 114. This state is shown in FIG. 1C which will further be described below in more detail.

The medical device 112 comprises the at least one transcutaneously insertable portion (not shown) and at least one housing 162 which is shown in FIG. 1C.

The inserter 164 may have an inserter housing 166. The inserter housing 166 may be configured to attach to the first side 122 of the mounting device 114. Specifically, the inserter housing 166 may be configured to fit into the collar 144 only with very little play. The inserter 164 may comprise at least one button 168. By pressing the button 168 towards the mounting device 114 such as illustrated with arrow 170 an insertion mechanism may be triggered. Thus, the transcutaneously insertable portion may be placed at least partially within the skin site of the user or the patient.

FIG. 1C shows the holder 110. With regard to details on the holder 110 reference can be made to the description of FIG. 1A above. Further, in FIG. 1C, the medical device 112 is depicted. Specifically, the kit 160 is shown.

In FIG. 1C, the housing 162 of the medical device 112 is shown. The housing 162 may be attachable to the first side 126 of the mounting device 114, specifically to the surface 130. When triggering the insertion mechanism by pressing the button 168 of the inserter 164 such as illustrated in FIG. 1B, the housing 162 may be moved from an interior of the inserter 164 to the surface 130 of the mounting device. After removing the inserter 164 from the mounting device 114 the medical device 112 may stay on the mounting device 114. For this purpose, the medical device 112 may have at least one attachment component (not shown) which is capable of connecting the medical device 112 to the first side 122 of the mounting device 114, such as at least one adhesive surface.

The housing 162 may specifically be an electronics unit 174 having at least one component for one or more of performing a measurement with the sensor, performing a voltage measurement, performing a current measurement, recording sensor signals, storing measurement signals or measurement data, transmitting signals or measurement data to another device. The electronics unit 174 may specifically be embodied as a transmitter or may comprise a transmitter, for transmitting data.

The housing 162 may have an opening 176, specifically a through opening 178. The housing 162 may be placed on the first side 122 such that the opening 176 lies in the center of the hole 138 of the mounting device 114 such as illustrated in FIG. 1A. Thus, an insertion aid of the inserter 164 such as an insertion cannula (not shown) may be removed from the body tissue after insertion via the opening 176.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LIST OF REFERENCE NUMBERS

110 holder
112 medical device
114 mounting device
116 strap
118 first strap
120 second strap
122 first side
124 second side
126 front side
128 rear side
130 flat surface
132 rough surface
134 adhesive material
138 hole
140 through hole
142 center
144 collar
146 protrusion
148 circumferential collar
150 side
152 strip
154 looped band
156 hole
158 through hole
160 kit
162 housing
164 inserter
166 inserter housing
168 button
170 arrow
174 electronics unit
176 opening
178 through opening

What is claimed is:

1. A holder for a medical device that has a transcutaneously insertable portion and a housing, the holder comprising:
a mounting device having a thickness of 0.1 mm to 2 mm and being configured for connection to the housing of the medical device, the mounting device having a first side and an opposing second side and having a hole reaching from the first side to the second side; and
two straps respectively attached to the mounting device and fixedly connectable to each other.

2. The holder according to claim 1, wherein the mounting device has a thickness of 0.3 mm to 1 mm.

3. The holder according to claim 1, wherein the mounting device is at least partially made of a rigid material.

4. The holder according to claim 1, wherein the mounting device comprises a collar that forms a protrusion on the first side.

5. The holder according to claim 4, wherein the collar is a circumferential collar.

6. The holder according to claim 4, wherein the collar is configured for centering the transcutaneously insertable portion of the medical device.

7. The holder according to claim 1, wherein at least one of the two straps is an elastic strap.

8. The holder according to claim 1, wherein the two straps are fixedly connectable to each other by at least one of a button, a snap, a hook-and-eye-closure, a magnetic locking, a side release buckle, and a zipper.

9. The holder according to claim 1, wherein at least one of the two straps is made of at least one foamed material selected from the group consisting of: a silicone polymer, neoprene, polyurethane.

10. The holder according to claim 1, wherein the holder is configured for being wrapped around an extremity.

11. A kit, comprising:
the holder according to claim 1; and
a medical device having a transcutaneously insertable portion.

12. The kit according to claim 11, wherein the medical device is selected from the group consisting of a medication device for delivering at least one medication to a user and a medical device for detecting at least one analyte in a body fluid.

13. The kit according to claim 11, wherein the transcutaneously insertable portion is selected from the group consisting of a sensor and an infusion cannula.

14. The kit according to claim 11, wherein the medical device has a medical device housing, the medical device housing having an opening configured to receive the transcutaneously insertable portion.

15. The kit according to claim 14, wherein the opening is aligned with the hole when the medical device is attached to the holder.

16. The kit according to claim 11, wherein the medical device is configured for attachment to the holder by an inserter.

17. A method for mounting a medical device comprising at least one transcutaneously insertable portion on a skin site of an extremity of a user, the method comprising the following steps:
a) mounting the holder according to claim 1 on the skin site of the extremity of the user by fixedly connecting the two straps with each other;
b) placing an inserter having the medical device on the first side of the mounting device such that an end of the transcutaneously insertable portion and the hole are congruent with each other; and
c) inserting the transcutaneously insertable portion into the skin site through the hole of the mounting device.

18. The method according to claim 17, wherein the medical device is placed on the first side of the mounting device via the inserter unit, wherein the inserter unit is removed from the mounting device after insertion of the transcutaneously insertable portion into the skin site.

19. The method according to claim 17, wherein the medical device is attached to the holder via the inserter and remains attached to the mounting device when the inserter unit is removed from the mounting device.
